(19) United States
(12) Patent Application Publication
(10) Pub. No.: US 2025/0134878 A1
(43) Pub. Date: May 1, 2025

(54) COMBINATION OF CHEK1 INHIBITOR AND TOP1 INHIBITOR FOR TREATING COLORECTAL CANCER

(71) Applicants: Genome Research Limited, London (GB); Stichting Het Nederlands Kanker Instituut-Antoni Van Leeuwenhoek Ziekenhuis, Amsterdam (NL)

(72) Inventors: Elizabeth Coker, London (GB); Mathew Garnett, London (GB); Patricia Jaaks, London (GB); Daniel Vis, Amsterdam (NL); Lodewyk Wessels, Amsterdam (NL)

(73) Assignees: Genome Research Limited, London (GB); Stichting Het Nederlands Kanker Instituut-Antoni Van Leeuwenhoek Ziekenhuis, Amsterdam (NL)

(21) Appl. No.: 18/837,155

(22) PCT Filed: Feb. 10, 2023
(86) PCT No.: PCT/EP2023/053384

§ 371 Date: Aug. 8, 2024

(30) Foreign Application Priority Data
Feb. 11, 2022 (GB) 2201825.3

(51) Int. Cl. A61K 31/4535; A61K 31/4745; A61K 31/497; A61K 31/519; A61K 31/5377; A61P 35/00

(52) U.S. Cl. CPC A61K 31/4535; A61K 31/4745; A61K 31/497; A61K 31/519; A61K 31/5377; A61P 35/00

(57) ABSTRACT

The present invention relates to a combination therapy a CHEK1 inhibitor and a TOP1 inhibitor for use in a method of treatment of colorectal cancer in a patient.

### FIELD OF THE INVENTION

The invention relates to combination therapies for the treatment of cancer.

### BACKGROUND

Despite advances in treatment cancer continues to have a major impact on societies, families, and individuals across the world. Cancer is among the leading causes of death worldwide. According to statistics provided by the National Cancer Institute, in 2018 there were 18.1 million new cases and 9.5 million cancer-related deaths worldwide. By 2040 the number of new cancer cases per year is expected to rise to 29.5 million and the number of cancer-related deaths to 16.4 million. Bowel cancer is one of the most common types of cancer.

The use of single-agent targeted therapies in patients with molecularly-defined tumours is improving cancer treatment. Nonetheless, many patients still lack effective treatments and pre-existing or acquired resistance limits the clinical benefit of even our most advanced medicines. Combination therapies using the growing number of targeted anti-cancer agents have potential to overcome resistance, to enhance response to existing drugs, to reduce dose limiting single agent toxicity, and to expand the range of treatments for patients.

### SUMMARY OF THE INVENTION

The invention is directed to the use of therapeutic combinations of active ingredients for the treatment of bowel cancer in patients, and especially to the combination of a CHEK1 inhibitor and a TOP1 inhibitor, suitably a camptothecin. As described herein, the inventors have observed synergy for combinations of a CHEK1 inhibitor with a TOP1 inhibitor in cancer cell lines.

In a first aspect, the invention may provide a combination of a CHEK1 inhibitor and a TOP1 inhibitor for use in a method of treatment of colorectal cancer in a patient. The TOP1 inhibitor irinotecan is of considerable interest for the treatment of bowel cancer but is unfortunately often associated with severe toxicities, especially neutropenia and diarrhoea. Data generated by the inventors and described herein suggest that the combination of irinotecan with a CHEK1 inhibitor may facilitate the use of irinotecan in a dose sparing or fractional dose approach, opening up irinotecan-based therapies to colorectal cancer patients for who established camptothecin-based regimens have been discontinued or are contraindicated. Accordingly, the invention may provide a camptothecin derivative (for example irinotecan) for use in a method of treating cancer in a patent wherein the camptothecin derivative is administered in a dose-sparing regimen together with administration of a CHEK1 inhibitor. “Dose-sparing” in this context will be understood to refer to administration of a camptothecin derivative in a total dose that is lower than that used in in previously approved or trialled treatment regimens.

The inventors further postulate that the combination may lead to a better response, namely greater tumour regression and/or increased survival, at currently used clinical doses.

In some cases, the invention may provide a CHEK1 inhibitor for use in a method of treatment of colorectal cancer in a patient, and wherein the CHEK1 inhibitor is administered to the patient in combination with TOP1 inhibitor, wherein the TOP1 inhibitor is a camptothecin derivative.

In some cases, the invention may provide a TOP1 inhibitor for use in a method of treatment of colorectal cancer in a patient, and wherein the TOP1 inhibitor is administered to the patient in combination with CHEK1 inhibitor, wherein the TOP1 inhibitor is a camptothecin derivative.

The inventors have identified the combination of a TOP1 inhibitor and a CHEK1 inhibitor as a potent combination in microsatellite stable (MSS) and/or KRAS-TP53 double mutant colon cancer cells, the combination driving cell apoptosis and enhancing response when compared to irinotecan alone.

Accordingly, in some embodiments the colorectal cancer is KRAS-TP53 double mutant colorectal cancer. The cancer may be, but is not necessarily, microsatellite stable (that is, the cancer may be microsatellite instable).

In some embodiments the colorectal cancer is microsatellite stable. The cancer may be, but is not necessarily, KRAS-TP53 double mutant colorectal cancer.

In some embodiments the colorectal cancer is KRAS-TP53 double mutant and microsatellite stable colorectal cancer.

Suitably, the TOP1 inhibitor is a camptothecin, for example selected from irinotecan, SN-38, topotecan, and camptothecin. A preferred TOP1 inhibitor is irinotecan or an active metabolite thereof. SN-38 is an active metabolite of irinotecan. That is, SN-38 is a preferred CHEK1 inhibitor. Another preferred CHEK1 inhibitor is camptothecin.

In some embodiments, the CHEK1 inhibitor is selected from rabusertib, SAR-020106, AZD7762, prexasertib, MK-8776, CCT245737, CHIR-124, PF-477736, VX-803, GDC-0575, ESP-01, and BEBT-260. In some embodiments, the CHEK1 inhibitor is selected from rabusertib, SAR-020106, AZD7762, prexasertib, and MK-8776. A preferred CHEK1 inhibitor is rabusertib.

It will be understood that the CHEK1 inhibitor and the TOP1 inhibitor may be administered together or separately and may be administered at the same time or at different times. For example, the compounds may be administered on different days as part of a treatment cycle or treatment regimen. Suitably but not necessarily the CHEK1 inhibitor and the TOP1 inhibitor will be formulated separately. For example, each or either compound may be formulated for oral or parenteral administration.

The combination therapies claimed may be used both curatively and palliatively. They may lead to better patient outcomes and/or experiences when compared to other treatment regimens and additionally or alternatively may expand the treatment options available to patients.

Suitably, the patient may be a human patient.

The invention also relates to a method of treatment of colorectal cancer in a patient in need thereof, wherein the method comprises the step of administering a combination of an effective amount of a CHEK1 inhibitor together with an effective amount of a TOP1 inhibitor to the patient, wherein the TOP1 inhibitor is a camptothecin derivative.

The invention also relates to a method of treatment of colorectal cancer in a patient in need thereof, wherein the method comprises the step of administering an effective amount of a CHEK1 inhibitor to the patient in combination with an effective amount of a TOP1 inhibitor, wherein the TOP1 inhibitor is a camptothecin derivative.

The invention also relates to a method of treatment of colorectal cancer in a patient in need thereof, wherein the method comprises the step of administering an effective amount of a TOP1 inhibitor to the patient in combination with an effective amount of a CHEK1 inhibitor, wherein the TOP1 inhibitor is a camptothecin derivative.

The invention also relates to a use of a combination of a CHEK1 inhibitor together with a TOP1 inhibitor in the manufacture of a medicament for the treatment of colorectal cancer in a patient, wherein the TOP1 inhibitor is a camptothecin derivative.

The invention also relates to a use of a CHEK1 inhibitor in the manufacture of a medicament for the treatment of colorectal cancer in a patient in combination with a TOP1 inhibitor, wherein the TOP1 inhibitor is a camptothecin derivative.

The invention also relates to a use of a TOP1 inhibitor in the manufacture of a medicament for the treatment of colorectal cancer in a patient in combination with a CHEK1 inhibitor, wherein the TOP1 inhibitor is a camptothecin derivative.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows synergy and activity of the combination of camptothecin and AZD7762 in (A) colon cancer cells and (B) in different colon cancer populations. See Example 1 for further details.

FIG. 2 shows the synergy and activity of reference combination AZD7762 and 5-FU in colon cancer cells. (A) compares MSI and MSS populations. (B) looks at the influence of TP53 mutation in a KRAS mutant population. See Example 2 for further details.

FIG. 3 shows ΔEₘₐₓ and ΔIC₅₀ values for CHEK inhibitors in combination with camptothecin. See Example 3 for further details.

FIG. 4 shows siRNA and Western Blot results for combinations of CHEK inhibitors and camptothecin: (A) Pooled siRNA against CHEK1, CHEK2 or PLK1 (positive cell death control)+/−SN-38 (TOP1). Read-out: viability (CTG); (B) Western blot of (A) confirming knockdown of CHEK1 and CHEK2; (C) Individual siRNAs against CHEK1 (positive cell death control)+/−SN-38 (TOP1). Read-out: viability (CTG); (D) Western blot of (C) confirming knockdown of CHEK1. See Example 3 for further details.

FIG. 5 shows colony formation for a claimed combination and the compounds used individually. See Example 4 for further details.

FIG. 6 shows a snapshot of the cell death signal recorded at 72 h for the combination of rabusertib and a TOP1i. See Example 5 for further details.

FIG. 7 shows PARP cleavage and cell death for the combination of rabusertib and a TOP1i in SW837 and SNU-81 cells. See Example 6 for further details.

FIG. 8 shows (A) shows tumour volume change over time for irinotecan and rabusertib compared to the agents alone; (B) shows tumour volume change at endpoint; (C) shows survival of SNU-81 engrafted mice after end of treatment. See Example 7 for further details.

FIG. 9 shows the effect of CHEK1i and TOPi on DNA double strand breaking, apoptosis and proliferation in tumours. See Example 8 for further details.

FIG. 10 shows the effect of siCHEK1 on the IC₅₀ of SN-38. See Example 9 for further details.

FIG. 11 shows the sensitivity of colorectal cancer cell lines to CHEK1 and topoisomerase inhibitor combinations. Colorectal cell lines were treated with the topoisomerase inhibitor SN-38 plus a CHEK1 inhibitor as indicated on each dot plot. The synergy metrics, Bliss window and HSA window, were generated as described in Example 10. Each point is a single replicate, with 5-8 replicates per cell line.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### CHEK1 Inhibitors

Checkpoint kinase 1 is commonly referred to in the literature as CHEK1. Checkpoint kinases (CHEKs) are involved in the DNA damage response in many cancer cells, and CHEK1 inhibition is associated with stalled DNA replication, replication fork collapses, and accumulation of DNA damage.

The term CHEK1 inhibitors (also referred to as CHEK1i) refers to compounds that have a half maximal inhibitory concentration (IC₅₀) of <15 nM in a cell-free assay, such as <10 nM. In some embodiments, the CHEK1 inhibitors have a half maximal inhibitory concentration (IC₅₀) of <15 UM in a cell-based assay, such as <10 μM, to induce cell-cycle arrest, or to inhibit phopho-CHEK1 levels, or induce DNA-damage in cells. For example, rabusertib has an in vitro IC₅₀ of 7 nM, based on an in vitro biochemical assay using recombinant CHEK1 kinase (van Ark-Otte et al., Br J Cancer, 1998 June; 77 (12): 2171-2176).

The inventors have observed that the combination response is CHEK1-specific. The compounds may also inhibit CHEK2, in which case they may be referred to as CHEK1/2i. Preferably, the combination comprises a CHEK1-specific inhibitor.

CHEK1 inhibitors include rabusertib, SAR-020106, AZD7762, prexasertib, MK-8776, CCT245737, CHIR-124, PF-477736, VX-803, GDC-0575, ESP-01, and BEBT-260. In some embodiments, CHEK1 inhibitors include rabusertib, SAR-020106, AZD7762, prexasertib, and MK-8776.

A preferred CHEK1i is rabusertib.

### Rabusertib

Rabusertib is also known as LY2603618 and IC-83. It is a highly selective CHEK1i with an IC₅₀ of 7 nM in a cell-free assay and has the following structure:

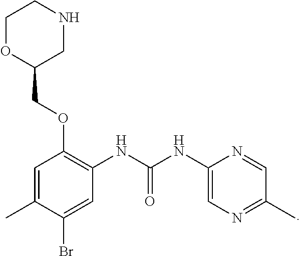 [SMILES: Cc1cnc(NC(=O)Nc2cc(Br)c(C)cc2OC[C@@H]2CNCCO2)cn1]

In IUPAC nomenclature it may be called 1-[5-bromo-4-methyl-2-[[(2S)-morpholin-2-yl]methoxy]phenyl]-3-(5-methylpyrazin-2-yl) urea. It is commercially available.

### SAR-020106

SAR-020106 is an ATP-competitive, potent, and selective CHEK1 inhibitor with an IC₅₀ of 13.3 nM a cell-free assay and good cellular activity. It has the following structure:

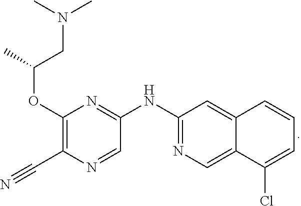 [SMILES: C[C@H](CN(C)C)Oc1nc(Nc2cc3cccc(Cl)c3cn2)cnc1C#N]

In IUPAC nomenclature it may be called 5-(8-chloroisoquinolin-3-ylamino)-3-((R)-1-(dimethylamino) propan-2-yloxy) pyrazine-2-carbonitrile. It is commercially available.

### AZD7762

AZD7762 is a CHEK1/2 inhibitor. It has an IC₅₀ against CHEK1 of 5 nM in a cell-free assay and has the following structure:

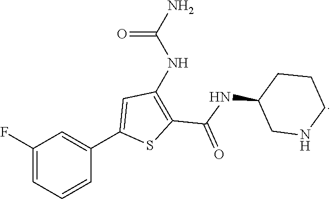 [SMILES: NC(=O)Nc1cc(-c2cccc(F)c2)sc1C(=O)N[C@H]1CCCNC1]

In IUPAC nomenclature it may be called(S)-5-(3-Fluorophenyl)-N-(piperidin-3-yl)-3-ureidothiophene-2-carboxamide. It is commercially available.

### Prexasertib

Prexasertib is also known as LY2606368 and ACR 368. It is a CHEK1/2 inhibitor and has an IC₅₀ against CHEK1 of 1 nM in a cell-free assay. It has the following structure:

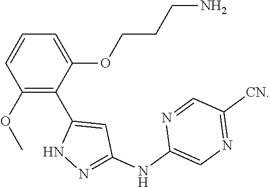 [SMILES: COc1cccc(OCCCN)c1-c1cc(Nc2cnc(C#N)cn2)n[nH]1]

In IUPAC nomenclature it may be called 5-[[5-[2-(3-aminopropoxy)-6-methoxyphenyl]-1H-pyrazol-3-yl]amino]pyrazine-2-carbonitrile. It is commercially available.

### MK-8776

MK-8776 is also known as SCH 900776 and is a selective CHEK1 inhibitor with an IC₅₀ of 3 nM in a cell-free assay. It shows 500-fold selectivity against CHEK2. It has the following structure:

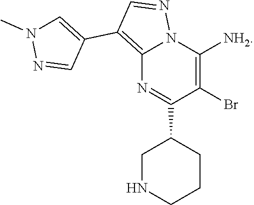 [SMILES: Cn1cc(-c2cnn3c(N)c(Br)c([C@@H]4CCCNC4)nc23)cn1]

In IUPAC nomenclature it may be called 6-bromo-3-(1-methyl-1H-pyrazol-4-yl)-5-[(3R)-piperidin-3-yl]pyrazolo[1,5-a]pyrimidin-7-amine. It is commercially available.

### CCT245737

CCT245737 is also known as SRA737 or PNT-737 and is an orally active CHEK1 inhibitor with an IC₅₀ of 1.4 nM. It exhibits >1,000-fold selectivity against CHEK2. It has the following structure:

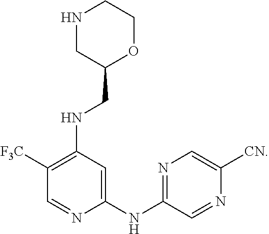 [SMILES: N#Cc1cnc(Nc2cc(NC[C@H]3CNCCO3)c(C(F)(F)F)cn2)cn1]

In IUPAC nomenclature it may be called 5-methyl-N2-(5-methylpyrazin-2-yl)-N4-[[(2R)-morpholin-2-yl]methyl]pyridine-2,4-diamine. It is commercially available.

### CHIR-124

CHIR-124 is a potent CHEK1 inhibitor with an IC₅₀ of 0.3 nM in a cell-free assay. It shows 2,000-fold selectivity against CHEK2. It has the following structure:

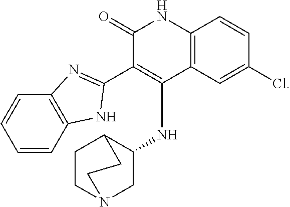 [SMILES: O=c1[nH]c2ccc(Cl)cc2c(N[C@@H]2CN3CCC2CC3)c1-c1nc2ccccc2[nH]1]

In IUPAC nomenclature it may be called 3-(1H-benzimidazol-2-yl)-6-chloro-4-[[(3S)-quinuclidin-3-yl]amino]-1H-quinolin-2-one. It is commercially available.

### PF-477736

PF-477736 is also known as PF-736 or PF-00477736. It is a selective, potent and ATP-competitive CHEK1 inhibitor. It has a Kᵢ (dissociation constant) of 0.49 nM in a cell-free assay and an IC₅₀ of 0.49 nM. It shows a ˜100-fold selectivity for CHEK1 than CHEK2. It has the following structure:

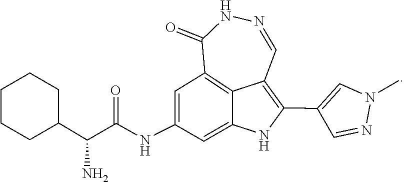 [SMILES: Cn1cc(-c2[nH]c3cc(NC(=O)[C@H](N)C4CCCCC4)cc4c3c2C=NNC4=O)cn1]

In IUPAC nomenclature it may be called (2R)-2-amino-2-cyclohexyl-N-[2-(1-methylpyrazol-4-yl)-9-oxo-3,10,11-triazatricyclo[6.4.1.0^{4.13}]trideca-1,4,6,8 (13), 11-pentaen-6-yl]acetamide. It is commercially available.

### VX-803

VX-803 is also known as M4344. It is an ATP-competitive, orally active, and selective inhibitor of ataxia telangiectasia and Rad3 related (ATR) kinase with Kᵢ of <150 pM. It is thought to inhibit ATR-driven phosphorylated CHEK1 (P-CHEK1) phosphorylation with IC₅₀ of 8 nM in a cell-free assay. It has the following structure:

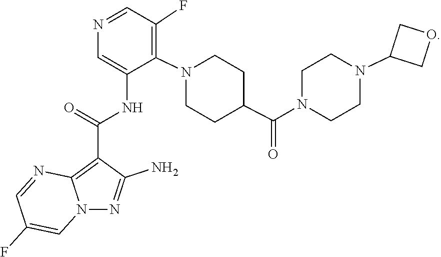 [SMILES: Nc1nn2cc(F)cnc2c1C(=O)Nc1cncc(F)c1N1CCC(C(=O)N2CCN(C3COC3)CC2)CC1]

In IUPAC nomenclature it may be called 2-amino-6-fluoro-N-[5-fluoro-4-[4-[4-(oxetan-3-yl) piperazine-1-carbonyl]-1-piperidyl]-3-pyridyl]pyrazolo[1,5-a]pyrimidine-3-carboxamide. It is commercially available.

### GDC-0575

GDC-0575 is also known as ARRY-575 or RG7741. It is a potent and selective CHEK1 inhibitor with an IC₅₀ of 1.2 nM. It has the following structure:

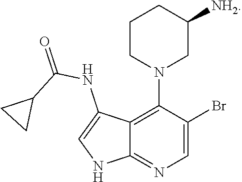 [SMILES: N[C@@H]1CCCN(c2c(Br)cnc3[nH]cc(NC(=O)C4CC4)c23)C1]

In IUPAC nomenclature it may be called N-[4-[(3R)-3-amino-1-piperidyl]-5-bromo-1H-pyrrolo[2,3-b]pyridin-3-yl]cyclopropanecarboxamide. It is commercially available.

### ESP-01

ESP-01 (Esperas Pharma Inc) is also known as LY 2880070. It is a selective CHEK1 inhibitor and is under development for the treatment of solid tumours including metastatic colorectal cancer, epithelial ovarian cancer, endometrial cancer, soft tissue sarcoma, gastrointestinal stromal tumours (GIST), pancreatic cancer and triple-negative breast cancer (TNBC). It is administered by the oral route and is in various clinical trials.

### BEBT-260

BEBT-260 (Guangzhou BeBetter Medicine Technology Co Ltd) is a CHEK1 inhibitor. Relevant patents include EP3411036.

TOP1 inhibitors

DNA topoisomerase I is commonly referred to in the literature as TOP1. It controls and alters the topologic states of DNA during transcription and is an interesting target for oncology.

TOP1 inhibitors previously used for clinical treatment include irinotecan and camptothecin. These are members of the class of compounds based on camptothecin, collectively referred to as the camptothecins or camptothecin derivatives. That is, a camptothecin derivative refers to camptothecin itself or a derivative thereof based on the core camptothecin motif. Camptothecin derivatives include irinotecan, topotecan, belotecan, lurtotecan, exatecan, gimatecan, and sinotecan. Alkylated, alkoxylated and hydroxylated derivatives have been reported in the literature. Accordingly, the TOP1 inhibitor may be selected from irinotecan, topotecan, belotecan, lurtotecan, exatecan, gimatecan, and sinotecan, and alkylated, alkoxylated and hydroxylated derivatives thereof.

The inventors have observed that the combination response may be TOP1-specific. The compounds may also inhibit TOP2, in which case they may be referred to as TOP1/2i. Preferably, the combination comprises a TOP1-specific inhibitor.

Suitably, the TOP1 inhibitor used in the methods of the present invention is a campothecin derivative, for example selected from irinotecan, topotecan, and camptothecin, or an active metabolite therefore such as SN-38 (a metabolite of irinotecan). Preferably, the TOP1 inhibitor is camptothecin or SN-38. Even more preferably, the TOP1 inhibitor is SN-38.

### Camptothecin

Camptothecin is also known as NSC-100880, CPT, Campathecin and (S)-(+)-Camptothecin. It is a specific inhibitor of TOP1 with an IC₅₀ of 0.68 UM in a cell-free assay. Its structure is:

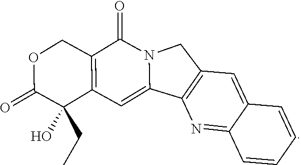 [SMILES: CC[C@@]1(O)C(=O)OCc2c1cc1n(c2=O)Cc2cc3ccccc3nc2-1]

In IUPAC nomenclature it may be called (19S)-19-ethyl-19-hydroxy-17-oxa-3,13-diazapentacyclo[11.8.0.0^{2.11}.0^{4.9}.0^{15,20}]henicosa-1 (21),2,4,6,8,10,15 (20)-heptaene-14,18-dione. It is commercially available.

### Irinotecan

Irinotecan is also known as CPT-11 and (+)-irinotecan. It is a topoisomerase I inhibitor for LoVo cells and HT-29 cells with IC₅₀ of 15.8 UM and 5.17 UM, respectively (van Ark-Otte et al., Br J Cancer, 1998 June; 77 (12): 2171-2176). Irinotecan is converted to SN-38 by carboxylesterases, which is the active metabolite. The structure of irinotecan is:

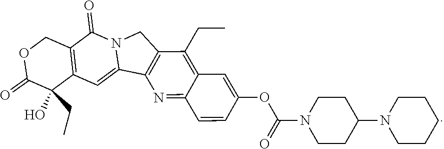 [SMILES: CCc1c2c(nc3ccc(OC(=O)N4CCC(N5CCCCC5)CC4)cc13)-c1cc3c(c(=O)n1C2)COC(=O)[C@]3(O)CC]

In IUPAC nomenclature it may be called [(19S)-10,19-diethyl-19-hydroxy-14,18-dioxo-17-oxa-3,13-diazapentacyclo[11.8.0.0^{2.11}.0^{4.9}.0^{15,20}]henicosa-1 (21),2,4(9),5,7,10,15(20)-heptaen-7-yl] 4-(1-piperidyl) piperidine-1-carboxylate. It is commercially available, for example as the hydrochloride salt.

### Topotecan

Topotecan, also known as Hycamtin and Potactasol, is an antineoplastic agent used to treat ovarian cancer that works by inhibiting DNA topoisomerases, in particularly DNA topoisomerase I. Its structure is:

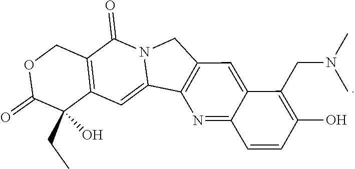 [SMILES: CC[C@@]1(O)C(=O)OCc2c1cc1n(c2=O)Cc2cc3c(CN(C)C)c(O)ccc3nc2-1]

In IUPAC nomenclature it may be called(S)-10-[(dimethylamino)methyl]-4-ethyl-4,9-dihydroxy-1H-pyrano[3′,4′:6,7]indolizino[1,2-b]quinoline-3, 14 (4H, 12H)-dione or (19S)-8-[(dimethylamino)methyl]-19-ethyl-7,19-dihydroxy-17-oxa-3,13-diazapentacyclo[11.8.0.0^{2.11}.0^{4.9}.0^{15,20}]henicosa-1(21),2(11),3,5,7,9,15(20)-heptaene-14,18-dione. It is commercially available, for example as the hydrochloride salt (which may be called Topotecan HCl, Toporan, NSC609699, Nogitecan, or SKFS 104864A).

### SN-38

SN-38 (also known as NK012) is also commercially available and its structure is:

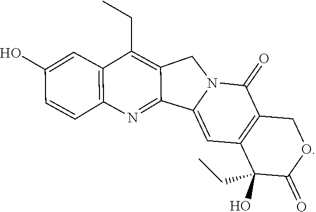 [SMILES: CCc1c2c(nc3ccc(O)cc13)-c1cc3c(c(=O)n1C2)COC(=O)[C@]3(O)CC]

SN-38 is an active metabolite of irinotecan, and it inhibits DNA topoisomerase I, DNA synthesis and causes frequent DNA single-strand breaks. SN-38 also induces autophagy. In IUPAC nomenclature it may be called (19S)-10,19-diethyl-7,19-dihydroxy-17-oxa-3,13-diazapentacyclo[11.8.0.0^{2.11}.0^{4.9}.0^{15,20}]henicosa-1(21),2,4(9),5,7,10,15(20)-heptaene-14,18-dione.

### Specific CHEK1 and TOP1 Inhibitor Combinations

In some cases, the combination of a CHEK1 inhibitor together with a TOP1 inhibitor for use in a method of treatment described herein may be specific CHEK1 inhibitor and TOP1 inhibitor combinations.

In some cases, the combination may be selected from: AZD7762 (CHEK1 inhibitor) and camptothecin (TOP1 inhibitor); MK-8776 (CHEK1 inhibitor) and SN-38 (TOP1 inhibitor); prexasertib (CHEK1 inhibitor) and SN-38 (TOP1 inhibitor); rabusertib (CHEK1 inhibitor) and SN-38 (TOP1 inhibitor); SAR-020106 (CHEK1 inhibitor) and SN-38 (TOP1 inhibitor); and CCT245737 (CHEK1 inhibitor) and SN-38 (TOP1 inhibitor).

In some cases, the combination for use in a method of treatment described herein may be the combination of AZD7762 (CHEK1 inhibitor) and camptothecin (TOP1 inhibitor). In some cases, the combination for use in a method of treatment described herein may be the combination of MK-8776 (CHEK1 inhibitor) and SN-38 (TOP1 inhibitor). In some cases, the combination for use in a method of treatment described herein may be the combination of prexasertib (CHEK1 inhibitor) and SN-38 (TOP1 inhibitor). In some cases, the combination for use in a method of treatment described herein may be the combination of rabusertib (CHEK1 inhibitor) and SN-38 (TOP1 inhibitor). In some cases, the combination for use in a method of treatment described herein may be the combination of SAR-020106 (CHEK1 inhibitor) and SN-38 (TOP1 inhibitor). In some cases, the combination for use in a method of treatment described herein may be the combination of CCT245737 (CHEK1 inhibitor) and SN-38 (TOP1 inhibitor).

### Pharmaceutically Acceptable Salts

As described herein, any compound may be provided as a pharmaceutically acceptable salt, hydrate or solvate. Suitable pharmaceutically acceptable salts are known in the art and are described in, for example, in Berge et al., J Pharm Sci, 1977 66(1) p 1.

### Administration of Active Ingredients

Compounds used in the methods of the invention may be administered by any suitable route, including oral and intravenous routes. It will be understood that oral administration may be preferred. The compounds may be provided in pharmaceutical compositions comprising the compound and one or more pharmaceutically acceptable excipients. Formulation for oral administration may be in the form of a tablet or a capsule comprising a powder or liquid.

Administration is preferably in a “therapeutically effective amount” or an “effective amount” (used interchangeably), this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Dosage regimens known in the art for the active ingredients described herein may also be used in the present invention.

For example, rabusertib has previously been administered parentally at 150 mg/m², 170 mg/m² or 230 mg/m² in clinical trials. See, for example, Weiss et al. Invest New Drugs 2013 31(1): 136-44 (PMID: 22492020), ClinicalTrials.gov Identifier: NCT01341457, Scagliotti et al. Invest New Drugs, 27 Jun. 2016, 34(5):625-635 (PMID: 27350064).

For irinotecan as a monotherapy, the recommended dose is 125-350 mg/m² administered as an intravenous infusion. The dose depends on whether it is a weekly or two or three weekly regimen. When used in combination with Leucovorin Calcium (LV) and Fluorouracil (5-FU), the dose is 125-180 mg/m² depending on the regimen. See, for example, https://www.pfizermedicalinformation.com/en-us/camptosar/dosage-admin, https://www.medicines.org.uk/emc/product/481/smpc#gref, and https://reference.medscape.com/drug/camptosar-irinotecan-342252.

Accordingly, the active ingredients described herein may be administered in dosages of about 1 mg to about 1000 mg, such as about 5 mg to about 500 mg, such as about 10 mg to about 400 mg. In some embodiments, the CHEK1 inhibitor (such as rabusertib) may be administered in a dosage of about 1 mg to about 1000 mg, such as about 50 mg to about 500 mg, such as about 100 mg to about 300 mg, such as about 170 mg to about 230 mg. In some embodiments, TOP1 inhibitor (such as camptothecin, such as irinotecan) may be administered in a dosage of about 1 mg to about 1000 mg, such as about 50 mg to about 500 mg, such as about 100 mg to about 400 mg, such as about 150 mg to about 350 mg.

As a dose sparing approach, the CHEK1 inhibitor (such as rabusertib) may be administered at full dose (such as 230 mg) and the concentration of the TOP1 inhibitor (such as camptothecin, such as irinotecan) may be reduced 2-10 fold the full dose. For example, the full dose of irinotecan is 350 mg/m². The 2-fold reduction of the full dose of irinotecan may be in the range of about 75 mg/m² to about 175 mg/m²; and the 10-fold reduction of the full dose of irinotecan may be in the range of about 15 mg/m² to about 35 mg/m².

The active ingredients described herein may be administered simultaneously or sequentially. In some embodiments of the combination therapy described herein, the CHEK1 and TOP1 inhibitors are administered sequentially. In some embodiments, the TOP1 inhibitor (such as camptothecin, such as irinotecan) would be administered first, followed by the CHEK1 inhibitor (such as rabusertib).

Each of the CHEK1 and TOP1 inhibitors may be independently administered orally or parentally. In some embodiments, the CHEK1 inhibitor (such as rabusertib) may be administered parentally, such as intravenously. In some embodiments, the TOP1 inhibitor (such as camptothecin, such as irinotecan) may be administered parentally, such as intravenously.

The active ingredients described herein may be administered at different times within a prescribed dosing cycle. The active ingredients described herein may be administered daily, such as once daily (QD), twice daily (BID), three times daily (TID), or four times daily (QID), or on a less frequent or intermittent schedule.

Suitably, the patient may be a human patient.

Combination with Other Chemotherapeutic Agents

Compounds used in the methods of the invention may be administered together with one or more other active agents, such as other chemotherapeutic agents, for the treatment of colorectal cancer. For example, the combination therapies as described herein may be used in combination with other chemotherapeutic agents such as a platinum-based chemotherapy, or with gemcitabine, or with 5-FU, or with 5-FU used in combination with Leucovorin Calcium. In such cases, the combination therapy (i.e. CHEK1 and TOP1 inhibitors) and the additional active agent(s) may be given together or separately, e.g. as constituents in the same pharmaceutical composition or formulation, or as separate formulations.

### Cancer Type

The invention relates to methods for the treatment of cancer in patients, and in particular the treatment of bowel (colorectal) cancer. Accordingly, in some aspect the invention relates to the treatment of colon cancer in a patient.

Suitably, the colorectal cancer is microsatellite stable (MSS). That is, the tumour is categorized as MSS by genomic profiling and/or immunohistochemistry. Tumours which are not MSS are termed MSI-High (Microsatellite Instability—High). Genomic profiling to determine MSI/MSS status is described in Kawakami et al., Curr. Treat. Options Oncol., 2015 July; 16 (7): 30 (PMID: 26031544) and testing systems are commercially available from, for example, Promega® (OncoMate™ MSI Dx Analysis System).

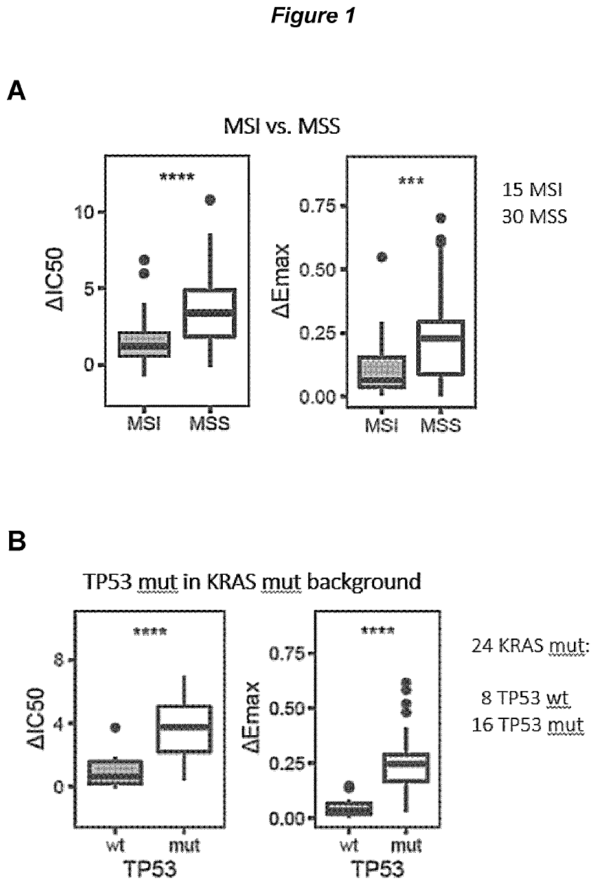

As can be seen from Example 1 and specifically FIG. 1a, synergy and activity is pronounced in this patient population. That is, in colorectal cancer patients identified as having the MSS tumour biomarker. Estimates of the proportion of colorectal tumours that are MSS varies, but it is widely agreed that over half of colorectal tumours are MSS.

In some embodiments, the colorectal cancer is KRAS-TP53 double mutant colon cancer. In other words, the tumour is categorized as having both the KRAS mutation and the TP53 mutation by genomic profiling.

The KRAS (Kirsten rat sarcoma virus) gene is an oncogene. KRAS mutation is thought to be associated with about 40% of colorectal cancers and may be determined by tests that are known in the field. Most methods include the use of PCR to amplify the appropriate region of the KRAS gene, including exons 2 and 3, and then utilize different methods to distinguish wild-type from mutant sequences in key codons, such as 12 and 13. The detection methods include nucleic acid sequencing, allele-specific PCR methods, single-strand conformational polymorphism analysis, melt-curve analysis, and probe hybridization. Tests for detection for KRAS mutations, such as Cobas® KRAS Mutation Test (Roche) and Therascreen KRAS RGQ PCR Kit (Qiagen), are FDA approved (https://www.fda.gov/medical-devices/in-vitro-diagnostics/list-cleared-or-approved-companion-diagnostic-devices-in-vitro-and-imaging-tools).

Tumour protein p53 (TP53) is a gene that codes for a tumour suppressor protein, which regulates expression of genes involved in cell cycle arrest, apoptosis, senescence, DNA repair, and changes in metabolism. TP53 mutations are associated with Li-Fraumeni syndrome and may be determined by tests that are known in the field. Methods to detect and analyse TP53 mutations include PCR assays that look for mutations present in exons 2, 3, 4, 5, 6, 7, 8, 9, 10, and 11 of TP53 (https://www.fda.gov/medical-devices/in-vitro-diagnostics/list-cleared-or-approved-companion-diagnostic-devices-in-vitro-and-imaging-tools).

As shown in FIG. 1b, a significantly higher degree of potency and efficacy was observed in the KRAS-TP53 double mutant population.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1

### Synergy and Activity Rate in of the Combination in Colon Cancer

AZD7762 (CHEK1, CHEK2) was screened in combination with the TOP1 inhibitor camptothecin in colon (n=45) cancer cell lines. To screen efficiently, the inventors used a 2×7 concentration matrix, or “anchored” approach. The inventors screened each anchor compound at two optimised concentrations and a discontinuous 1,000-fold (7-point) dose-response curve of the library compound. Viability was read out after 72 h of drug treatment using CellTiter-Glo and drug responses for single agent and combination responses were fitted. Single-agent and combination viability measurements were fitted per cell line and multiple parameters derived including: 1) anchor viability effect, 2) library and combination viability effect at the highest-used library concentration (library Eₘₐₓ and combo Eₘₐₓ), and 3) the estimated library drug concentration producing a 50% viability reduction (IC₅₀) for the library and combination. The inventors compared observed combination response of cells to the Bliss independence-predicted response based on monotherapy activity, and classified drug combinations based on shifts beyond Bliss in potency (ΔIC₅₀; i.e. increased sensitivity) or efficacy (ΔEₘₐₓ; i.e. reduced cell viability) (Bliss, Annals of Applied Biology, 1939, 26 (3), 585-615). The inventors classified combination-cell line pairs as synergistic if, at either anchor concentration, combination IC₅₀ or Eₘₐₓ was reduced 8-fold or 20% viability over Bliss, respectively. Good levels of synergy and activity were observed. See Table 1-Rate of Synergy in colon cancer cell lines.

TABLE 1

| Combination* | Colon |
|---|---|
| Camptothecin + AZD7762 (CHEKi) | 40% |
| AZD7762 (CHEKi) + camptothecin | 50% |

*the first compound listed is the anchor compound. The combination was tested in both orientations.

The synergy and activity rate also subset to MSS (n=30) and MSI (n=15), or KRAS mut (n=24) and KRAS wt (n=21) cell lines. Over 50% of MSS or KRAS mut colon cell lines show synergy and activity. See Table 2.

TABLE 2

| | MSS | MSI | KRAS mut | KRAS wt |
|---|---|---|---|---|
| Combination | colon | colon | colon | colon |
| Camptothecin + AZD7762 | 55% | 7% | 48% | 32% |
| AZD7762 + camptothecin | 63% | 27% | 46% | 55% |

Activity in MSI (n=15) and MSS (n=30) cell lines was compared (FIG. 1a). KRAS mutant (n=24) cell lines were investigated (n=8, TP53 wild type; n=16 TP53 mutant) (FIG. 1b). Microsatellite stability (MSS) or TP53 mutation in a KRAS mutant background sensitises colon cancer cells to treatment with AZD7762 and camptothecin. In both populations the inventors observed a significantly higher shift in potency (ΔIC₅₀) and in efficacy (ΔEₘₐₓ). Plotted are averaged combination responses. Box and whisker plots show the median and interquartile range. A two-sided Welch's t-test was performed (*=p≤0.05, **=p≤0.01, ***=p≤0.001, ****=p≤0.0001).

### Example 2

Comparison with Other Combinations

AZD7762 (CHEK1, CHEK2) was screened in combination with chemotherapeutics in colon (n=45) cancer cell lines. Viability was read out after 72 h of drug treatment using CellTiter-Glo and drug responses for single agent and combination responses were fitted. Synergy was called based on significant shifts in efficacy (ΔEₘₐₓ) or potency (ΔIC₅₀) over the expected drug combination response [as calculated by Bliss; synergy if ΔEₘₐₓ≥ 20% viability or ΔIC_{50≥ 3} (=8-fold concentration shift)]. Activity was called based on significant shifts of viability reduction of the combination compared to single agent activity (combination showed 20% more viability reduction than both single agents). As can be seen, the synergy and activity of the claimed combination in colon cancer cell lines is higher than the combination of the CHEK1 inhibitor AZD7762 with other chemotherapeutic agents (Table 3).

TABLE 3

| | 5-FU (DNA | Oxaliplatin | Vinorelbine |
|---|---|---|---|
| Combination | antimetab.) | (DNA alkyl.) | (Microtub.) |
| AZD7762 (CHEKi) = Library | 28% | 2% | 4% |
| AZD7762 (CHEKi) = Anchor | 26% | 0% | 0% |

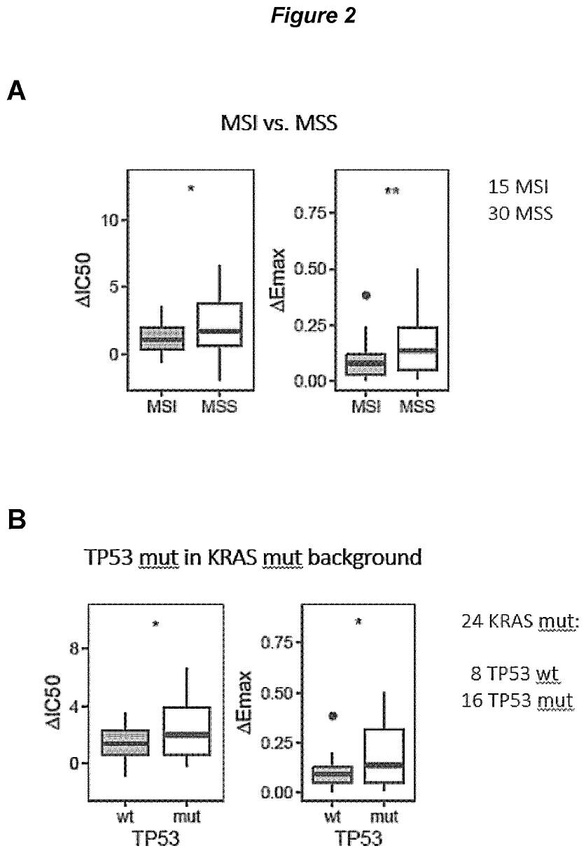

The combination of AZD7762 and 5-FU was further investigated (FIG. 2a, FIG. 2b). Microsatellite stability (MSS) or TP53 mutation in a KRAS mutant background only slightly sensitises colon cancer cells to treatment with AZD7762+5-FU. In both populations the inventors observed a significant, but only slightly higher shift in potency (ΔIC₅₀) and in efficacy (ΔEₘₐₓ). Plotted are averaged combination responses. Box and whisker plots show the median and interquartile range. A two-sided Welch's t-test was performed (*=p≤0.05, **=p≤0.01, ***=p≤0.001, ****=p≤0.0001).

### Example 3

### CHEK1 Specificity

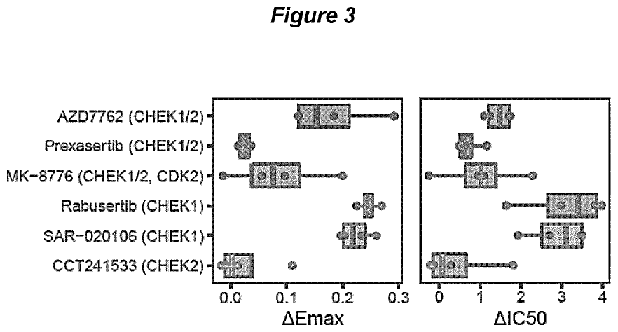

To test CHEK specificity the inventors seeded SW620, SW837, SNU-81 or LS-1034 cells in 96-well plates (770-2,750 cells per well) and treated them with camptothecin (anchor, 0.025 UM) in combination with six CHEK inhibitors with different specificities (libraries, all dosed at 1 μM highest used concentration unless indicated): AZD7762 (CHEK1, CHEK2), prexasertib (CHEK1, CHEK2), MK-8776 (CHEK1, CHEK2, CDK2), SAR-020106 (CHEK1), rabusertib (CHEK1) and CCT241533 (CHEK2; 2 UM). After 96 h of drug treatment viability was measured with CellTiter-Glo® 2.0 (CTG; Promega). Drug response curves were fitted and combination response metrics ΔEₘₐₓ and ΔIC₅₀ calculated by comparing the observed combination response with the expected (Bliss) combination response. The higher the ΔEₘₐₓ and ΔIC₅₀, the stronger the combination response. SW620, SW837, SNU-81 and LS-1034 are all MSS and KRAS-TP53 double mutant colon cancer cell lines. CHEK1-selective inhibitors SAR-020106 and rabusertib produced large shifts in potency (median ΔIC₅₀: 8.5-10.5-fold shift) and efficacy (median ΔEₘₐₓ: 0.22-0.24) in combination with camptothecin in 4 cell lines, whereas a CHEK2-selective inhibitor CCT-241533 did not (FIG. 3).

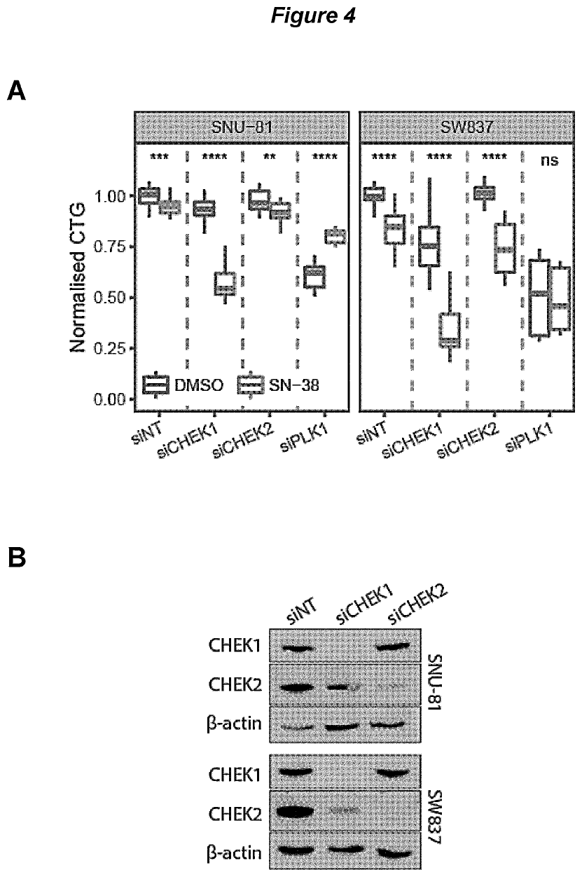

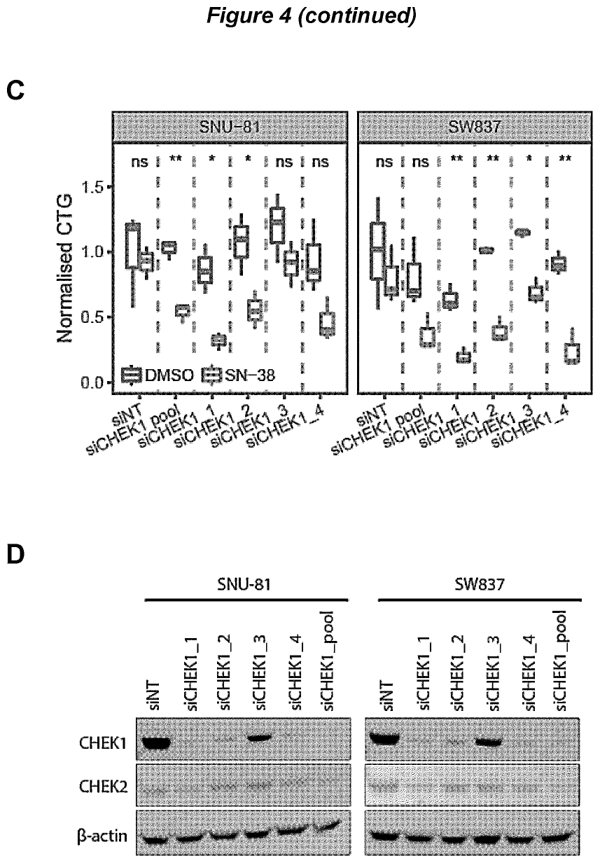

siRNA experiments were also performed (FIGS. 4a and 4c). FIG. 4a shows A) Pooled siRNA against CHEK1, CHEK2 or PLK1 (positive cell death control)+/−SN-38 (TOP1). Read-out: viability (CTG). FIG. 4c shows individual siRNAs against CHEK1 (positive cell death control)+/−SN-38 (TOP1). Read-out: viability (CTG).

For siRNA experiments, SW837 and SNU-81 cells (8,000 and 16,000 cells per well, respectively) were reverse transfected with siRNAs of a non-targeting pool as negative control (siNT; Dharmacon, D-001810-10-5), polo-like kinase 1 (PLK1) pool as positive control (Dharmacon, L-003290-00-0010), CHEK1 pool (Dharmacon, L-003255-00-0005) or four individual CHEK1 siRNAs (LQ-003255-00-0005), and CHEK2 pool (Dharmacon, L-003256-00-0005) using lipofectamine RNAiMax (Thermofisher). After 30 h, 0.025 UM or a dose range of 0.001-9 UM SN-38 or DMSO were added and viability was measured 72 h later with CTG. Signal was normalised to siNT+DMSO controls. Statistical significance between conditions was tested using a Student's t-test, *=p≤0.05, **=p≤0.01, ***=p≤0.001, ****=p≤0.0001. FIGS. 4a and 4c confirm that the effect of the TOP1+CHEK combination is primarily mediated through inhibition of CHEK1.

Western blots analysis was also done to confirm gene knockdown (FIGS. 4b and 4d). SW837 or SNU-81 cells were reverse transfected with siNT, siCHEK1 or siCHEK2 as described above. Cells were collected 72 h after transfection and lysed in RIPA buffer (Sigma-Aldrich, R0278). Cell lysates were separated by SDS-PAGE, transferred onto PVDF membranes and probed for anti-CHEK1 (Santa Cruz Biotechnology, sc-8408, 1:200; mouse), anti-CHEK2 (Cell Signaling Technologies, D9C6, 1:1000; rabbit), and anti-β-actin (Abcam, ab6276, 1:5000; mouse) as a loading control. FIG. 4b confirms knockdown of CHEK1 and CHEK2. FIG. 4d confirms knockdown of CHEK1.

### Example 4

### Combination Compared to Single Agents

Cells (from cell lines LS-1034, SW837, and SNU-81) were seeded in 6-well plates at 50,000 cells per well. Drugs (0.1 nM SN-38, 0.5 UM rabusertib, 0.5 μM CCT241533) or DMSO were added on day 1 and were refreshed through medium change on day 8. 14 days after drug treatment start, the cells were fixed in 4% paraformaldehyde (Sigma-Aldrich) in PBS (10 minutes at room temperature) and stained with Giemsa (10%; Sigma-Aldrich) for at least 30 minutes at room temperature.

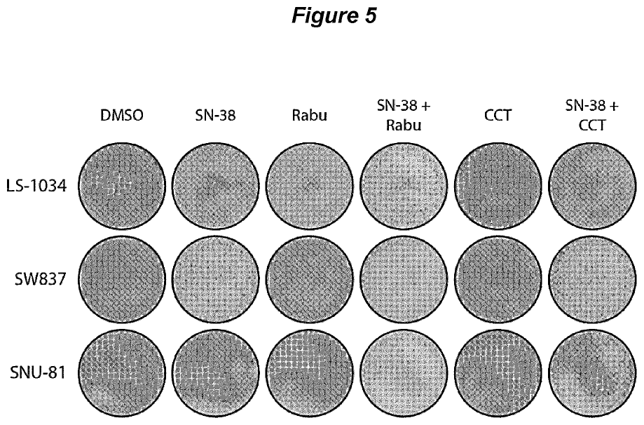

As can be seen from FIG. 5, in three separate cell lines tested the combination of rabusertib (CHEK1) and TOP1i (SN-38) leads to less colony formation than either single-agent, or when TOP1i is combined with CCT241533 (CHEK2 selective inhibitor).

The inventors note that quantification of the number of colonies formed is challenging, due to their small size. This is often dependent on the cell line used.

### Example 5

### Rabusertib (CHEK1i) and TOP1i Leads to Cell Death Beyond Additive Response

Cells were seeded in 96-well plates (typically 5,000-16,000 cells per well). After 24 h drugs (0.125 μM staurosporine (positive control), 0.025 μM SN-38, 0.75 μM rabusertib, 0.75 μM CCT241533) or DMSO and real-time fluorescent reagents for detection of cell death (CellTox™ Green; 1:1000; Promega) or caspase-3/7 activity (IncuCyte® Caspase-3/7 Red; 1:1000; Essen Bioscience) were added. Pictures were recorded every 2 h for 96 h using an Incucyte (Essen Bioscience). Recorded fluorescent signals were measured as mean intensity per cell area and normalised to time 0 h. Mean of 3-4 biological replicates.

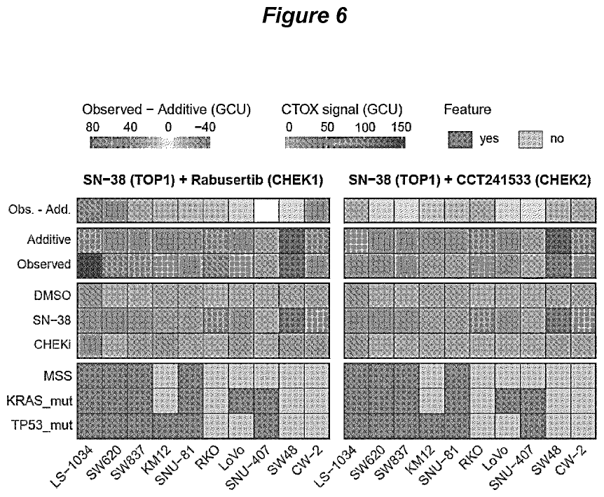

A snapshot of the cell death signal recorded at 72 h has been converted into the plot shown in FIG. 6. GCU is an abbreviation for green calibrated units. The top panels (the three rows Obs.—Add.; Additive; and Observed; with the Observed-Additive (GCU) scale as the key) show the additive responses (sum of SN-38 and CHEKi single-agent responses; Additive row) and experimentally observed responses (Observed row), and the delta is the difference between observed and additive responses (Obs.—Add. row). The middle panel (the three rows DMSO; SN-38; and CHEKi; with the CTOX signal (GCU) scale as the key) shows the effect on cell death of the individual drugs, with DMSO as a control in absence of either drug. The bottom panel (the three rows MSS; KRAS_mut; and TP53_mut; with the Feature colouring as the key) shows the mutational status for KRAS and TP53, and microsatellite stability (MSS) status in the cell lines (where the cell lines are identified along the bottom).

As can be seen from FIG. 6, CHEK1i but not CHEK2i in combination with TOP1i leads to increased cell death beyond additive response in many colon cancer cell lines, particularly those that are MSS or KRAS-TP53 double mutant.

### Example 6

### PARP Cleavage to Determine Cell Death

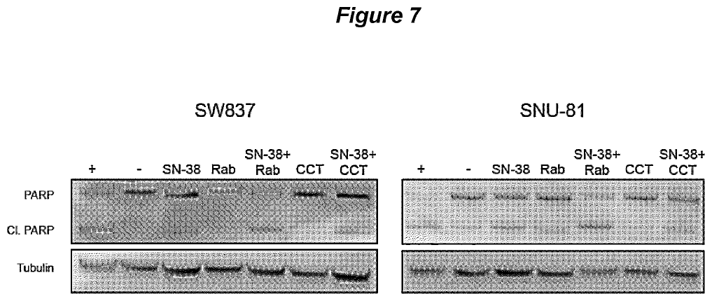

SW837 (1 million) or SNU-81 (1.5 million) cells were seeded in 10 cm dishes and treated with drugs (0.025 nM SN-38, 1.5 UM rabusertib, 1.5 μM CCT241533, 2 μM MG-132 (positive control)) or DMSO the day after. After 72 h alive and dead cells were collected and lysed in RIPA buffer (Sigma-Aldrich) supplemented with 1 mM DTT (Cayman Chemicals) and protease and phosphatase inhibitors (Roche). Cell lysates were separated by SDS-PAGE, transferred onto PVDF membranes and probed for anti-PARP (Cell Signalling Technologies, 9542, 1:1,000; rabbit) and anti-β-tubulin (Sigma-Aldrich, T4026, 1:5,000; mouse) as loading control. The results are shown in FIG. 7, confirming that the combination leads to increased cell death beyond the individual drugs.

### Example 7

### Effect of CHEK1i and TOPi on Tumour Volume Change or Survival

4.5×10⁶ LS-1034 cells, 5×10⁶ SW837 cells or 2.5×10⁶ SNU-81 cells in 30% matrigel were injected subcutaneously into the right flank of 6-week-old NOD/SCID mice. Once tumours reached an average volume of ˜300-400 mm³, mice were randomised into treatment arms, with: LS-1034: n=6 mice for vehicle, n=11 for irinotecan, n=12 for rabusertib and irinotecan+rabusertib; SW837: n=6 mice for vehicle and rabusertib, n=8 for irinotecan, n=4 for irinotecan+rabusertib. SNU-81: n=5 mice for vehicle and irinotecan+rabusertib, n=6 for rabusertib, n=10 for irinotecan.

Rabusertib was administered orally, 200 mg/kg daily (vehicle: 16.66% Captisol®, CyDex Inc, in 25 mM phosphate buffer, pH 4); irinotecan was administered intraperitoneally, 25 mg/kg twice a week (vehicle: physiological saline). Tumour size was evaluated once weekly by calliper measurements, and the approximate volume of the mass was calculated using the formula 4/3π·(d/2)²·D/2, where d is the minor tumour axis and D is the major tumour axis. Results were considered interpretable when a minimum of four mice per treatment group reached the prespecified endpoints (at least 3 weeks on therapy or development of tumours with average volumes larger than 1500 mm³ within each treatment group in trials aimed to assess drug efficacy; at least 3 weeks after treatment cessation or development of individual tumours with volumes larger than 750 mm³ in survival experiments aimed to assess tumour control by therapy). Operators were blinded during measurements. In vivo procedures and related biobanking data were managed using the Laboratory Assistant Suite49. Animal procedures were approved by the Italian Ministry of Health (authorization 806/2016-PR).

Statistical significance for tumour volume changes during treatment was calculated using a two-way ANOVA. For endpoint comparisons, statistical analysis was performed by two-tailed unpaired Welch's t-test. Statistical analyses in the survival experiments were performed by log-rank (Mantel-Cox) test. For all tests, the level of statistical significance was set at p<0.05. Graphs were generated and statistical analyses were performed using the GraphPad Prism (v9.0) statistical package.

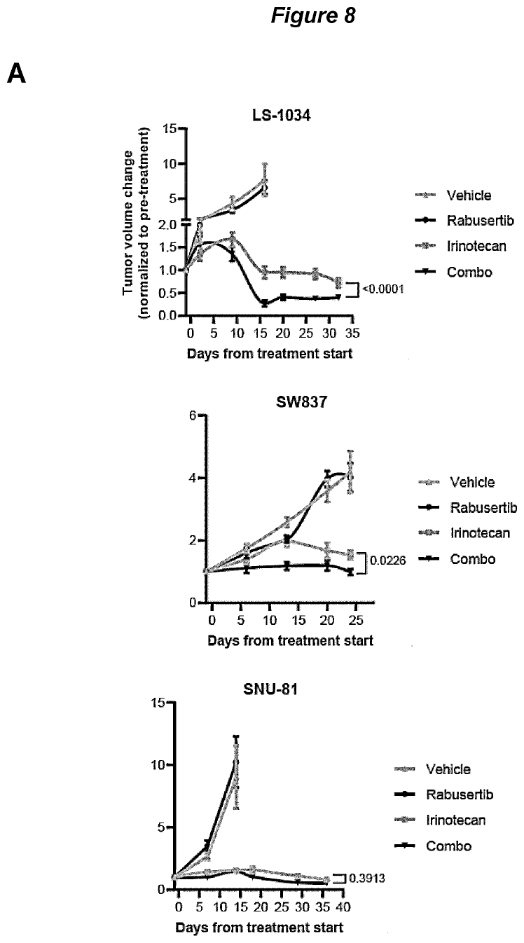

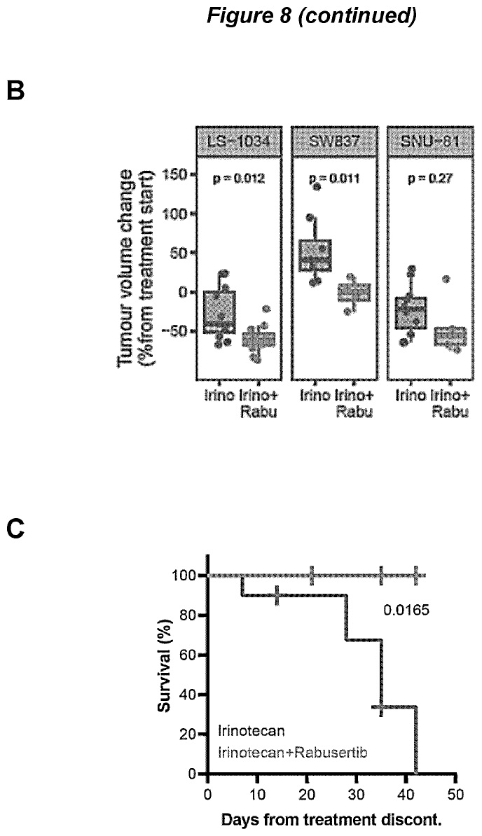

FIG. 8a shows tumour volume change over time. FIG. 8b shows tumour volume change at endpoint. FIG. 8c shows survival of SNU-81 engrafted mice after end of treatment. As can be seen from the figures, the effect of the combination therapy on end of-treatment tumour volumes and on tumour growth inhibition over time was more pronounced than irinotecan alone (Welch's t-test, p<0.05) (FIGS. 8a and 8b). In SNU-81, although combination therapy was more similar to irinotecan alone, the resumption of tumour growth after drug withdrawal was delayed in mice treated with the combination compared to those treated with irinotecan alone (log-rank Mantel-Cox test, endpoint 750 mm³), suggesting a fitness disadvantage (FIG. 8c).

### Example 8

### Effect of CHEK1i and TOPi on DNA Double Strand Breaking, Apoptosis and Proliferation in Tumours

Morphometric quantitation of Kᵢ67, active caspase-3 (active cas-3), and phospho-H2AX immunoreactivity was performed in LS-1034 xenografts from mice treated with vehicle (until tumours reached an average volume of 1500 mm³) or the indicated compounds (after 72 hours). Tumours (n=1-3 for each treatment arm) were explanted and subjected to histological quality check and immunohistochemical analysis with the following antibodies: mouse anti-Kᵢ-67 (MIB-1) (Dako #GA626), rabbit anti-cleaved caspase-3 (Asp175) (Cell Signaling #9661) and rabbit anti-phospho-histone H2AX (Ser139) (20E3) (Cell Signaling #9718). After incubation with secondary antibodies, immunoreactivities were revealed by DAB chromogen (Dako). Images were captured with the Leica LAS EZ software using a Leica DM LB microscope. Morphometric quantitation was performed by ImageJ software using spectral image segmentation. Software outputs were manually verified by visual inspection of digital images. Each dot represents the value measured in one optical field (40× for Kᵢ67 and phospho-H2AX; 20× for active caspase-3), with 2-10 optical fields (Kᵢ67 and phospho-H2AX) and 3-5 optical fields (active caspase-3) per tumour depending on the extent of section area (n=12-30 for Kᵢ67 and phospho-H2AX; n=10-15 for active caspase-3). The plots show means±SD. Mean+/−SD. Statistical analysis by two-tailed unpaired Welch's t-test.

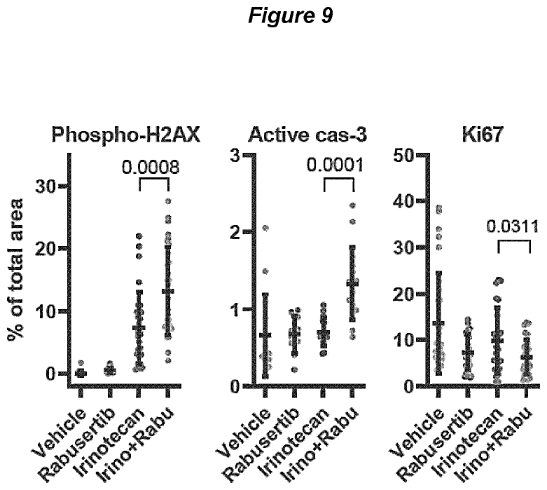

The results are shown in FIG. 9, which the combination leading to increased DNA double strand breaks (phospho-H2AX positive cells), more apoptotic cells (active caspase-3 positive cells) and fewer proliferative cells (reduced Kᵢ67 positive cells) in tumours in vivo.

### Example 9

Effect of siCHEK1 on IC₅₀ of TOP1i

SW837 and SNU-81 cells (8,000 and 16,000 cells per well, respectively) were reverse transfected with siRNAs of a non-targeting pool as negative control (siNT; Dharmacon, D-001810-10-5), CHEK1 pool (Dharmacon, L-003255-00-0005) or CHEK2 pool (Dharmacon, L-003256-00-0005) using lipofectamine RNAiMax (Thermofisher). After 30 h, a dose range of 0.001-9 UM SN-38 or DMSO were added and viability was measured 72 h later with CTG. Signal was normalised to siNT+DMSO controls. Mean±SD of three independent replicates.

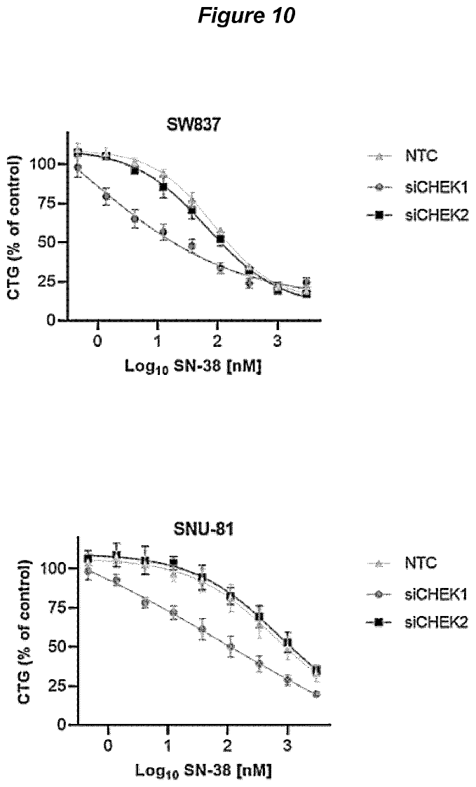

The results are shown in Table 4 and FIG. 10. siCHEK1 shifts the IC₅₀ of TOP1i, indicating that loss of CHEK1 sensitizes cells to TOP1i. These results are consistent with inhibition of CHEK1 leading to increased sensitivity to TOP1i, and suggest a combination therapy of CHEK1i and TOP1i could be used to reduce the dosage of TOP1i administered to patients in clinics, potentially reducing the clinical toxicity associated with TOP1 inhibition.

TABLE 4

| | siNT | siCHEK1 | siCHEK2 |
|---|---|---|---|
| IC₅₀ in SNU-81 | 611 nM | 84 nM | 715 nM |
| IC₅₀ in SW837 | 84 nM | 0.7 nM | 67 nM |

### Example 10

### Other CHEK1 and Topoisimerase Inhibitor SN-38 Combinations in Colon Cancer Cell Lines

### Method:

A screen was carried out in 3 colon cancer cell lines (CL-11, SNU-81 and SW837) using a 7×7 matrix approach generating 49 wells of data per cell line/drug combination. For each combination, one CHEK1 inhibitor was combined with SN-38, over a discontinuous 1,000-fold (7-point) dose range. Viability was measured after 72 h of drug treatment using CellTiter-Glo reagent. Single-agent and combination viability measurements were fitted per cell line and multiple parameters derived including single agent values and a range of synergy scores.

For all 49 concentration combination measurements a Bliss excess was calculated by comparing the observed combination response of cells to the Bliss independence-predicted response based on monotherapy activity. The “Bliss window” was reported as the highest mean Bliss excess value measured across the 25 possible 3×3 submatrices, or ‘windows’, across the 7×7 dose matrix.

In addition, a HSA (Highest Single Agent) excess was calculated for all 49 concentration combination measurements by comparing the observed combination response of cells to the highest single agent response for either Drug A or Drug B, whichever is highest. The “HSA window” was reported as the highest mean HSA excess value measured across the 25 possible 3×3 submatrices, or ‘windows’, across the 7×7 dose matrix.

### Results:

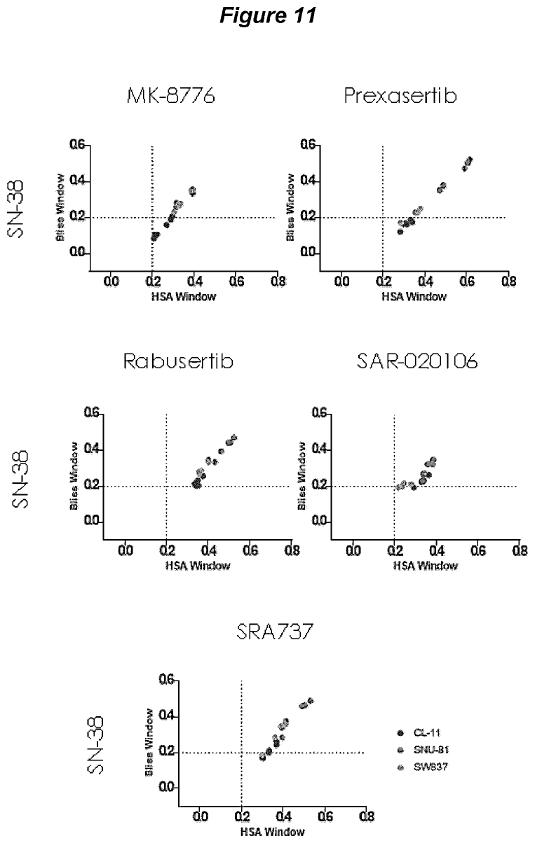

This screen comprised of SN-38 (a Topoisomerase I inhibitor) in combination with each of the following CHEK1 inhibitors: MK-8776, Prexasertib, Rabusertib, SAR-020106, SRA737. All three cell lines are MSS and KRAS/TP53 double mutant. Results show that synergy is high for SN-38 in combination with any of the CHEK1 inhibitors, as seen by high scores for the Bliss window and HSA window (FIG. 11).

### DISCUSSION

The inventors explain that the results show that the combination of a TOP1 inhibitor plus CHEK1 inhibition is synergistic in MSS or KRAS-TP53 double mutant colon cancer cells, leading to apoptosis and suppression of tumour xenograft growth.

Camptothecin is an analogue of the standard-of-care chemotherapeutic irinotecan used for treatment of colon cancer and CHEK1 inhibitors, including AZD7762, have been shown to potentiate responses of DNA-damaging compounds through abrogation of DNA damage-induced cell cycle arrest. Notably, the inventors observed that this combination yielded high synergy rates in MSS colon cancer cell lines (62.1% and 53.3% for both screening configurations) and showed significantly higher potency and efficacy in MSS cell lines than MSI cell lines (two-sided Welch's t-test, p<0.001, FIG. 1a). Furthermore, KRAS mutant colon showed high synergy for this combination (46% and 48%), and the inventors found that KRAS-TP53 double mutant cell lines had significantly stronger combination responses than KRAS single mutant cell lines (two-sided Welch's t-test, p<0.0001, FIG. 1b). Hence, the inventors identified two potential molecular contexts, MSS and KRAS-TP53 double mutant colon, with notable benefit from the combination of CHEKi and TOP1i.

To confirm the observed synergy and investigate whether synergy was dependent on inhibition of CHEK1, CHEK2 or both, the inventors combined camptothecin with six CHEK inhibitors with different specificities. CHEK1-selective inhibitors SAR-020106 and rabusertib led to large shifts in potency (median ΔIC₅₀: 8.5-10.5-fold shift) and efficacy (median ΔEₘₐₓ: 0.22-0.24) in combination with camptothecin when tested in 4 cell lines, whereas a CHEK2-selective inhibitor CCT241533 did not (FIG. 3). Furthermore, combining SN-38, the active metabolite of the TOP1 inhibitor irinotecan, with siRNA against CHEK1, but not CHEK2, resulted in a synergistic viability reduction (FIGS. 3 to 5). The addition of siCHEK1 but not siCHEK2 led to an at least 7-fold shift in IC₅₀ of SN-38 [SNU-81:7.2-fold (IC₅₀ SINT: 611.1 nM; siCHEK1: 84.4 nM; siCHEK2: 714.7 nM); SW837: 120-fold (IC₅₀ SINT: 84.4 nM; siCHEK1: 0.69 nM; siCHEK2: 66.6 nM)], suggesting that modulation of CHEK1 could be used to lower doses of TOP1 inhibitors. The results indicate that combination effects of TOP1i+CHEKi are primarily mediated through inhibition of CHEK1.

The inventors next evaluated the combination using colony formation assays. Combining low concentrations of SN-38 with rabusertib (CHEK1) resulted in fewer colonies formed and increased cell death (FIGS. 5 and 6) than either drug alone, or when combining SN-38 with CCT241533 (CHEK2). Cell death effects of SN-38+rabusertib surpassed an additive response for many colon cell lines, particularly in cell lines with weak to moderate response to SN-38 alone, consistent with CHEK1 inhibition potentiating the effect of TOP1 inhibition. The combination effect in colon cell lines ranged from less than additive to robust potentiation, with all MSS cell lines and most KRAS mutant cell lines showing at least additive response. We further found that the combination was associated with caspase 3/7 activation and PARP cleavage, both markers of apoptosis (FIG. 7).

To investigate the effects of the combination of TOP1i and CHEK1i on tumour growth in vivo, the inventors engrafted three colon cancer cell lines (LS-1034, SW837, SNU-81) in NOD/scid mice and treated them with irinotecan (TOP1), rabusertib (CHEK1), or with a combination of the two drugs. In LS-1034 and SW837, which showed more cell death of the combination of TOP1i and CHEK1i than SNU-81 in vitro (FIG. 6), the effect of the combination therapy on end-of-treatment tumour volumes and on tumour growth inhibition over time was more pronounced than that of irinotecan alone (Welch's t-test, p<0.05); in SNU-81, the impact of the combination therapy was more similar to that exerted by irinotecan alone (FIGS. 8a and b). However, resumption of SNU-81 tumour growth after drug withdrawal was delayed in mice treated with the combination compared to those treated with irinotecan alone (log-rank Mantel-Cox test, endpoint 750 mm³), suggesting a fitness disadvantage (FIG. 8c). Additionally, combination treatment led to more DNA double strand breaks (phospho-H2AX positive cells), less proliferative and more apoptotic tumour cells than observed in irinotecan treated LS-1034 tumours 72 h after treatment start (FIG. 9).

Taken together, the inventors believe that these data validate the combination of TOPi and CHEK1i as a potent combination in MSS and KRAS-TP53 double mutant colon cancer cells, which drives cell apoptosis and enhances response compared to irinotecan alone.

As proof-of-concept the inventions validated in vitro and in vivo an irinotecan and CHEK1i combination. While CHEK1i and DDA combinations have been linked to TP53 and KRAS, to the best of our knowledge, this is the first report of notable activity of CHEK1i in combination with chemotherapy in MSS and KRAS-TP53 double mutant colon. Clinical trials combining CHEKi with chemotherapy have demonstrated variable anti-tumour activity, particularly for unselected patients, and have been associated with toxicity. Since irinotecan is approved for the treatment of colon cancer, and rabusertib is a CHEK1-selective drug with an acceptable safety profile in Phase 1 clinical trials, these data indicate this combination, with appropriate consideration of potential toxicity, offers an effective treatment option for patients with MSS or KRAS-TP53 double mutant colon cancer. These represent populations with a presently unmet clinical need, and the inventors' observed synergy rates exceeding those of combinations previously used in clinical trials or presently the subject of ongoing clinical trials. For example, non-selective CHEK inhibitors have been tested clinically in combination with chemotherapeutics (notably Gemcitabine) in unselected patient populations and have largely been unsuccessful due to lack of efficacy or toxicity, or both. The inventors' observation indicates CHEK1 inhibition in combination with TOP1i in patients with MSS or KRAS-TP53 mutant colon cancer may be particularly responsive.

### REFERENCES

A number of publications are cited above in order to more fully describe and disclose the invention and the state of the art to which the invention pertains. Full citations for these references are provided below. The entirety of each of these references is incorporated herein.
Berge et al., J Pharm Sci, 1977 66(1) p 1
Bliss, Annals of Applied Biology, 1939, 26(3), 585-615
ClinicalTrials.gov Identifier: NCT01341457 EP3411036
Kawakami et al., Curr. Treat. Options Oncol., 2015 July; 16(7):30 (PMID: 26031544)
Remington's Pharmaceutical Sciences, 20th Edition, 2000, pub. Lippincott, Williams & Wilkins
Scagliotti et al. Invest New Drugs, 27 Jun. 2016, 34(5):625-635 (PMID: 27350064)
Weiss et al. Invest New Drugs 2013 31(1):136-44 (PMID: 22492020)
van Ark-Otte et al., Br J Cancer, 1998 June; 77(12): 2171-2176
https://www.fda.gov/medical-devices/in-vitro-diagnostics/list-cleared-or-approved-companion-diagnostic-devices-in-vitro-and-imaging-tools
https://www.medicines.org.uk/emc/product/481/smpc #gref
https://reference.medscape.com/drug/camptosar-irinotecan-342252

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

### Priority Clauses

1. A combination of a CHEK1 inhibitor and a TOP1 inhibitor for use in a method of treatment of colorectal cancer in a patient, wherein the TOP1 inhibitor is a camptothecin.
2. The combination for use according to clause 1, wherein the colorectal cancer is KRAS-TP53 double mutant colorectal cancer.
3. The combination for use according to clause 1, wherein the colorectal cancer is microsatellite stable.
4. The combination for use according to clause 1, wherein the colorectal cancer is KRAS-TP53 double mutant and microsatellite stable colorectal cancer.
5. The combination for use according to clause 4, wherein the camptothecin is selected from irinotecan, SN-38, topotecan, and camptothecin.
6. The combination for use according to clause 4, wherein the camptothecin is irinotecan.
7. The combination for use according to any preceding clause, wherein the CHEK1 inhibitor is selected from rabusertib, SAR-020106, AZD7762, prexasertib, MK-8776, CCT245737, CHIR-124, PF-477736, VX-803, GDC-0575, ESP-01, and BEBT-260.
8. The combination for use according to clause 7, wherein the CHEK1 inhibitor is selected from rabusertib, SAR-020106, AZD7762, prexasertib, and MK-8776.
9. The combination for use according to clause 8, wherein the CHEK1 inhibitor is rabusertib.
10. The combination for use according to any preceding clause, wherein the CHEK1 inhibitor and the TOP1 inhibitor are administered separately.

1. A combination of a CHEK1 inhibitor and a TOP1 inhibitor for use in a method of treatment of colorectal cancer in a patient, wherein the TOP1 inhibitor is a camptothecin derivative.

2. A CHEK1 inhibitor for use in a method of treatment of colorectal cancer in a patient, and wherein the CHEK1 inhibitor is administered to the patient in combination with TOP1 inhibitor, wherein the TOP1 inhibitor is a camptothecin derivative.

3. A TOP1 inhibitor for use in a method of treatment of colorectal cancer in a patient, and wherein the TOP1 inhibitor is administered to the patient in combination with CHEK1 inhibitor, wherein the TOP1 inhibitor is a camptothecin derivative.

4. The combination for use according to claim 1, the CHEK1 inhibitor for use according to claim 2, or the TOP1 inhibitor for use according to claim 3, wherein the colorectal cancer is KRAS-TP53 double mutant colorectal cancer.

5. The combination for use according to claim 1, the CHEK1 inhibitor for use according to claim 2, or the TOP1 inhibitor for use according to claim 3, wherein the colorectal cancer is microsatellite stable.

6. The combination for use according to claim 1, the CHEK1 inhibitor for use according to claim 2, or the TOP1 inhibitor for use according to claim 3, wherein the colorectal cancer is KRAS-TP53 double mutant and microsatellite stable colorectal cancer.

7. The combination, the CHEK1 inhibitor, or the TOP1 inhibitor for use according to any preceding claim, wherein the camptothecin derivative is selected from irinotecan, SN-38, topotecan, and camptothecin.

8. The combination, the CHEK1 inhibitor, or the TOP1 inhibitor for use according to claim 7, wherein the camptothecin derivative is irinotecan.

9. The combination, the CHEK1 inhibitor, or the TOP1 inhibitor for use according to claim 7, wherein the camptothecin derivative is camptothecin.

10. The combination, the CHEK1 inhibitor, or the TOP1 inhibitor for use according to claim 7, wherein the camptothecin derivative is SN-38.

11. The combination, the CHEK1 inhibitor, or the TOP1 inhibitor for use according to any preceding claim, wherein the CHEK1 inhibitor is selected from rabusertib, SAR-020106, AZD7762, prexasertib, MK-8776, CCT245737, CHIR-124, PF-477736, VX-803, GDC-0575, ESP-01, and BEBT-260.

12. The combination, the CHEK1 inhibitor, or the TOP1 inhibitor for use according to claim 11, wherein the CHEK1 inhibitor is selected from rabusertib, SAR-020106, AZD7762, prexasertib, and MK-8776.

13. The combination, the CHEK1 inhibitor, or the TOP1 inhibitor for use according to claim 11, wherein the CHEK1 inhibitor is rabusertib.

14. The combination, the CHEK1 inhibitor, or the TOP1 inhibitor for use according to any preceding claim, wherein the CHEK1 inhibitor and the TOP1 inhibitor are administered separately.
